(19) United States
(12) Patent Application Publication
(10) Pub. No.: US 2024/0033381 A1
(43) Pub. Date: Feb. 1, 2024

(54) QUANTITATIVE NIR REFERENCE AND EXPOSURE

(71) Applicant: ARTHREX, INC., Naples, FL (US)

(72) Inventors: Michael Dominik Steiner, Goleta, CA (US); Efrain O. Morales Correa, Santa Ynez, CA (US)

(21) Appl. No.: 18/227,049

(22) Filed: Jul. 27, 2023

Related U.S. Application Data
(60) Provisional application No. 63/392,587, filed on Jul. 27, 2022.

(51) Int. Cl. A61K 49/00; A61B 5/0275; A61B 5/02; A61B 5/00

(52) U.S. Cl. CPC A61K 49/0034; A61B 5/0275; A61B 5/02007; A61B 5/0071

(57) ABSTRACT

A method for fluorescent imaging of a subject, the method having the steps of: placing an illuminator in a surgical area of a subject, the illuminator having an emission band substantially similar to an fluorescence imaging agent; administering the fluorescence imaging agent to the subject; acquiring a plurality of images of the surgical area of the subject showing fluorescence intensity over a predetermined time period; and analyzing the plurality of images to determine changes in sensed fluorescence from the fluorescence imaging agent; wherein an exposure gain is applied to the images based on changes in detected intensity of the fluorescence from the illuminator.

### CROSS REFERENCE TO RELATED APPLICATION

This application claims priority of U.S. Provisional Patent Application No. 63/392,587, filed on Jul. 27, 2022, entitled QUANTITATIVE NIR REFERENCE AND EXPOSURE, the entire contents of which are hereby incorporated herein by reference.

### BACKGROUND

The present disclosure relates to surgical systems and methods and, more particularly, to surgical systems and methods of imaging.

During endoscopic surgical procedures, visualization agents, such as dyes, are often used to visualize tissue of interest. One use of dyes is to detect anastomotic leakage after intestinal resection. During this procedure, a surgeon injects a dye into an area of interest and monitors the levels of detected dye over time to assess leakage.

However, systems for measuring visualization agents are very sensitive to motion and typically require that the camera position, camera angle and the target anatomy be fixed in position. It is very difficult to fix all apparatus and anatomy in position in a clinical setting. Additionally, existing systems typically require immediate observation of emitted radiation from visualization agents or the procedure may need to be repeated.

There is a need for a system and method of measuring visualization agents that remedies the shortcomings of the prior art.

### SUMMARY

The present disclosure is directed to systems and methods of fluorescent imaging of a subject that remedy the shortcomings of the prior art. For example, the system and methods disclosed herein may allow a user to hold a camera in-hand without the need to fix all positions and may be capable of computing emitted radiation from visualization agents in real time.

In an implementation, a method is disclosed for fluorescent imaging of a subject that may include one or more steps of placing an illuminator in a surgical area of a subject, the illuminator having an emission band substantially similar to a fluorescence imaging agent; administering the fluorescence imaging agent to the subject; acquiring a plurality of images of the surgical area of the subject showing fluorescence intensity over a predetermined time period; and analyzing the plurality of images to determine changes in sensed fluorescence from the fluorescence imaging agent. An exposure gain may be applied to the images based on changes in detected intensity of the fluorescence from the illuminator.

Optionally, analyzing the images further comprises determining a perfusion slope of the fluorescence imaging agent. The perfusion slope may be a direct indication of blood flow in the surgical area of the subject. In an implementation, the surgical area may be a portion of the gastrointestinal tract of the subject and the perfusion slope may be an indication of leakage in the surgical area. Optionally, the fluorescence imaging agent comprises indocyanine green.

In an additional implementation, a method for fluorescent imaging of a subject comprising the steps of: placing a fluorescence reference in a surgical area of a subject, the fluorescence reference having an emission band substantially similar to a fluorescence imaging agent; administering the fluorescence imaging agent to the subject; acquiring a plurality of images of the surgical area of the subject showing fluorescence intensity over a predetermined time period; and analyzing the plurality of images to determine changes in sensed fluorescence from the fluorescence imaging agent. An exposure gain may be applied to the images based on changes in fluorescence detected from the fluorescence reference.

Optionally, analyzing the images further comprises determining a perfusion slope of the fluorescence imaging agent. The perfusion slope, may be a direct indication of blood flow in the surgical area of the subject. In an implementation, the surgical area may be a portion of the gastrointestinal tract of the subject and the perfusion slope may be an indication of leakage in the surgical area. Optionally, the fluorescence imaging agent comprises indocyanine green.

In an additional implementation, a method for fluorescent imaging of a subject comprising the steps of: administering a fluorescence imaging agent to the subject; acquiring a plurality of images of the surgical area of the subject showing visible light and fluorescence intensity over a predetermined time period; and analyzing the plurality of images to determine a changes in sensed fluorescence from the fluorescence imaging agent. An exposure gain of the fluorescence intensity images may be adjusted based on changes detected in the visible light images.

In an implementation, the autoexposure system provides substantially the same exposure gain adjustment for both the visible light images the fluorescence intensity images. Optionally, analyzing the images further comprises determining a perfusion slope of the fluorescence imaging agent. The perfusion slope may be a direct indication of blood flow in the surgical area of the subject. In an implementation, the surgical area may be a portion of the gastrointestinal tract of the subject and the perfusion slope may be an indication of leakage in the surgical area. Optionally, the fluorescence imaging agent comprises indocyanine green.

This disclosure is also directed to an endoscopic camera system for fluorescent imaging of a subject. In an implementation, the system comprises a camera further comprising an image sensor; a camera controller in communication with the camera, the camera controller further comprising a processor; and a light source coupled to the camera for illuminating a region of interest. The system also has at least one of the group consisting of: a fluorescence illuminator and a fluorescence reference configured for placement in the region of interest. The processor may be configured to acquire a plurality of images of the region of interest of the subject showing fluorescence intensity over a predetermined time period; analyze the plurality of images to determine changes in sensed fluorescence; and apply an exposure gain to the images based on changes in fluorescence detected from the fluorescence illuminator or the fluorescence reference.

The system may also comprise a fluorescence imaging agent. Optionally, the processor may further be configured to determine a perfusion slope of the fluorescence imaging agent. The fluorescence imaging agent and the fluorescence reference may be indocyanine green. The fluorescence imaging agent may be indocyanine green and the fluorescence illuminator may be configured to emit light in substantially the same wavelength band as indocyanine green.

These and other features are described below.

### BRIEF DESCRIPTION OF THE DRAWINGS

The features, aspects and advantages of the present disclosure will become better understood with regard to the following description, appended claims and accompanying figures wherein:

FIG. 1 is a schematic diagram of a system for fluorescent imaging of a subject using an illuminator according to an implementation;

FIG. 2 is a further schematic diagram of an endoscopic camera system according to an implementation;

FIG. 3 is a diagram illustrating exposure compensation in a system for fluorescent imaging of a subject using an illuminator according to an implementation;

FIG. 4 is a schematic diagram of a system for fluorescent imaging of a subject using a fluorescence reference according to an implementation;

FIG. 5 is a diagram illustrating exposure compensation in a system for fluorescent imaging of a subject using a fluorescence reference according to an implementation;

FIG. 6 is a graph illustrating perfusion of a fluorescence imaging agent in a patient;

FIG. 7 is a flowchart illustrating a method for fluorescent imaging of a subject using an illuminator according to an implementation;

FIG. 8 is a flowchart illustrating a method for fluorescent imaging of a subject using a fluorescence reference according to an implementation; and

FIG. 9 is a flowchart illustrating a method for fluorescent imaging of a subject using detected changes in visible light images to adjust exposure gain of detected fluorescence according to an implementation.

### DETAILED DESCRIPTION

In the following description of the preferred implementations, reference is made to the accompanying drawings which shows by way of illustration specific implementations in which the system and methods may be practiced. It is to be understood that other implementations may be utilized and structural and functional changes may be made without departing from the scope of this disclosure.

Quantitative fluorescence analysis methods involve measuring a baseline fluorescence intensity and observing how the fluorescence intensity changes in time. A camera that detects light in the fluorescence wavelength band can accurately measure fluorescence, provided that there is no subject motion, camera motion, or illumination changes. In typical endoscopic surgeries, these three complicating factors are present. The system and method may compensate for those factors and may be practiced through different preferred implementations.

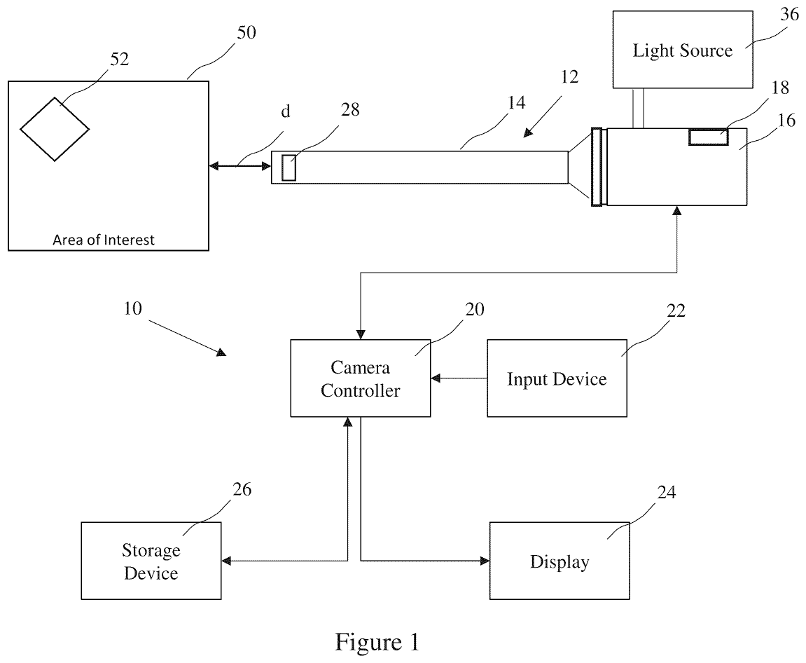

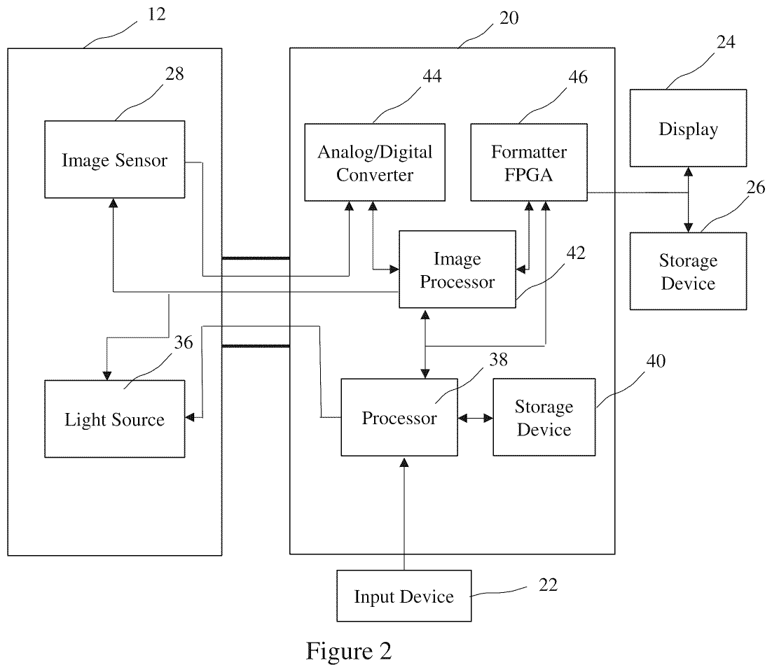

With reference to FIGS. 1 and 2, an endoscopic camera system 10 according to an implementation has a camera 12. The camera 12 may have a shaft 14 couplable to a handpiece 16. The handpiece 16 may have an input device 18, such as buttons, switches or dials. The handpiece 16 may be connectable to a camera controller 20 (“CCU” or “camera controller”). The handpiece 16 and the camera controller 20 may be connected via wire to facilitate data transfer between the camera and the camera controller. The camera 12 and the camera controller 20 may also be wirelessly connected to facilitate data transfer, such as via IEEE 802.11b or IEEE 802.11n or ultra-wide band (UWB). The camera controller 20 may be connectable to at least one input device 22 such as a mouse, keyboard, touchpad, or touchscreen monitor. Additionally, the camera controller 20 may be connectable to a display 24 and a storage device 26, such as for storing images.

An image sensor 28 may be positioned inside the shaft 14 and proximal to a distal tip 30 of the shaft 12. Additionally, the camera 12 may be coupled to a light source 36. The light source 36 may be inside of the camera 12. Additionally a first light source 36, such as a visible light source, may be inside of the camera 12 and a second light source having different wavelengths, such as a fluorescent excitation light source, may be coupled to the camera 12.

The light source 36 may include a lamp. The lamp may be, for example, a semiconductor light source such as laser or LED to illuminate the field of view. The light source 36 is configured to appropriately illuminate the field of view of the video camera. Further, the light generated as well as camera sensitivity may extend beyond the visible spectrum. The illumination may be intended to excite fluorescence directly in a target, or in a fluorescent substance such as indocyanine green, that is then sensed by the camera. For example, the light source 36 may produce illumination in the near infrared (NIR) range and the camera may sense the fluorescence at a longer IR wavelength. The illumination and camera sensitivity could extend from UV to NIR continuously or may be composed of separate narrow bands.

Referring to FIG. 2, the camera controller 20 may be a programmable unit containing sufficient processing capacity to accommodate a wide range of control, user interface and image acquisition/processing functions. The camera controller 20 may include a processor 38 that runs program applications providing for a variety of capabilities. For instance, an image capture and display capability may allow for both display of a live feed of an image through the display 24 coupled to the camera controller 20, as well as image capture. Captured images may be stored, such as in an internal storage device 40 or external storage device 26, or transmitted to other devices.

An image processor 42 may control and may process the output from the image sensor 28. Although other controllers and processors may be used to control and process the output from the image sensor 28, use of one or more FPGAs for processing video images may allow the system to achieve precise timing to generate a standard video output signal. User interface logic and possible external network connectivity may be performed by software running on the processor 38.

In an implementation, analog RGB data may be transmitted from the image sensor 28 to the camera controller 20. The Analog RGB data may pass through an Analog/Digital converter 44 to the image processor 42 where the video may be processed. The processed video may then be passed to a video output that may include a formatter FPGA 46 where the video may be formatted into various display formats. The formatter FPGA 46 may also overlay information, such as patient and/or doctor information, onto the video. The formatted video may be converted to an analog signal for display. The formatted video may be sent to the display 24 and/or the storage device 26. Alternatively, an Analog/Digital converter may be located in the camera head and digital RGB data may be transmitted from the camera head 12 to the camera controller 20. Additionally, the image sensors may include Analog/Digital converters.

The camera controller 20 may issue commands to the camera 12 to adjust its operating characteristics, and the camera 12 may send confirmation to the camera controller 20 that the camera received the commands. The image processor 42 and/or the processor 38 may communicate with a shutter driver either in the camera controller 20 or the camera 12 to control an exposure period of the image sensing module 28. Additionally, the image processor 42 and/or the processor 38 may communicate with the light source 36 to control the drive current to the lamp of the light source.

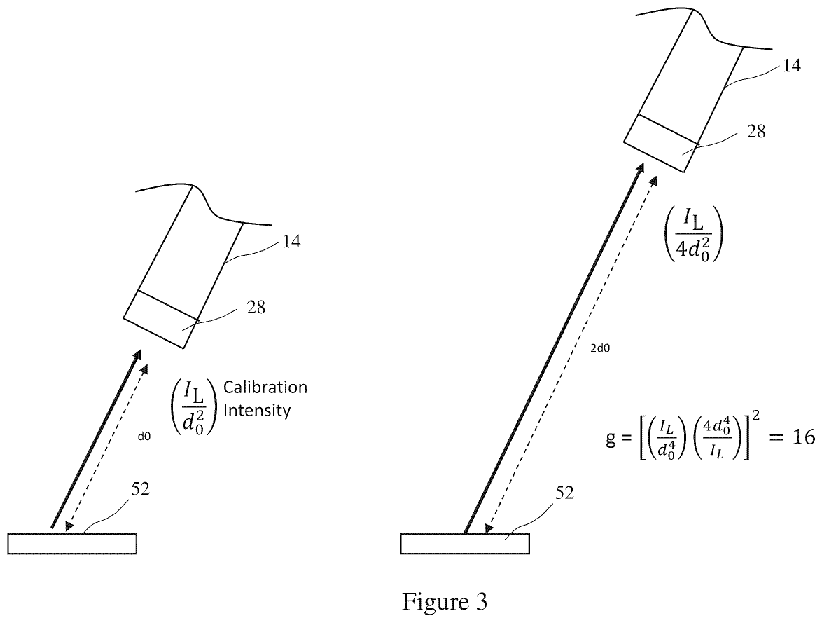

With reference to FIGS. 1 and 3, an endoscopic camera system 10 utilizing a fixed illuminator in-situ as a fluorescent emission reference will now be described. As shown in FIG. 1, the camera 12 may be positioned to visualize an area of interest 50. An illuminator 52 may be positioned within the area of interest 50. The illuminator 52 may be for example, an LED. The LED may be powered by a battery coupled to the LED. The illuminator 52 may be configured to emit light at a predetermined wavelength of a fluorescence imaging agent used in surgery. The fluorescence imaging agent may be, for example and without limitation, indocyanine green (ICG) dye or fluorescein dye and the illuminator 52 may be configured to emit light in the same wavelength as ICG or fluorescein.

The illuminator 52 may be single use or may be sterilizable. The illuminator 52 may be placed in the area of interest 50 using, for example, an endoscopic forceps, or other grasping device. The illuminator 52 may be fixed to the area of interest. The illuminator 52 may be fixed to the area of interest, such as by for example a pin or a needle. The illuminator 52 may lay on top of tissue in the area of interest. The illuminator 52 may be an attachment to or mounted in a surgical implement that is positioned in the area of interest. Multiple illuminators 52 may be used in an area of interest.

Once the camera 12 and the illuminator 52 are positioned in the area of interest 50, the location of the illuminator 52 within the captured images may be determined. The signal level of the image may be measured at the determined fixed illuminator 52 location. For illustration, the fixed illuminator 52 signal level is L₀ when the camera is not moving, the fixed illuminator is not moving, and the fixed illuminator is at a fixed distance from the camera. This signal level can be determined before signals from any fluorescence imaging agents used in surgery are measured. After this baseline illuminator signal level is measured, the fluorescence imaging agent signal measurement may begin, and the illuminator signal level at time t during endoscopy is Lₜ. The ratio

$${g}_{L} = {( \frac{{L}_{0}}{{L}_{t}} )}^{2}$$

is a gain that compensates for system exposure changes due to camera motion, tissue motion, and exposure control state changes when multiplied by the measured fluorescent dye signal.

FIG. 3 illustrates a change in gain when the camera is moved from a first position d₀ to a second position 2d₀ twice as far from the area of interest 50 and the fixed illuminator 52. With the use of a fixed illuminator 52, when the camera 12 is twice as far away, the distance from the excitation light source 36 to any fluorescence imaging agents used in surgery doubles which reduces the emitted fluorescence from the fluorescence imaging agents used in surgery, in addition to a reduction in detected emitted fluorescence as a result of increased distance. This is different than the effect on the fixed illuminator 52 which is not dependent on light from the excitation light source and does not change the amount of light emitted.

An equation for calculating the relevant gain for non-visible light exposure when the camera is moved from a first position d₀ to a second position 2d₀ twice as far from the area of interest 50 and the fixed illuminator 52 is:

$$g = {[ ( \frac{{I}_{L}}{{d}_{0}^{4}} ) ( \frac{4 {d}_{0}^{4}}{{I}_{L}} ) ]}^{2} = 16$$

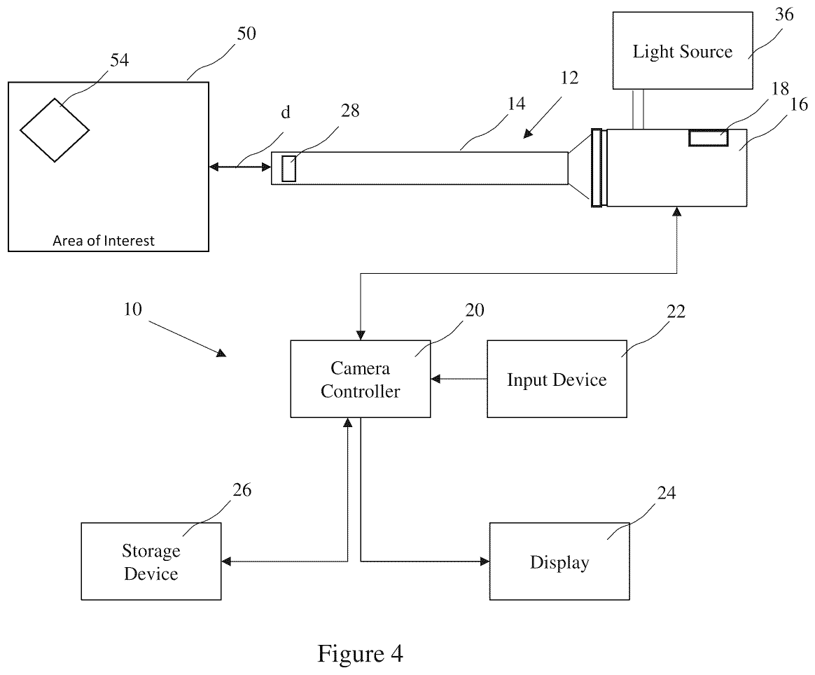

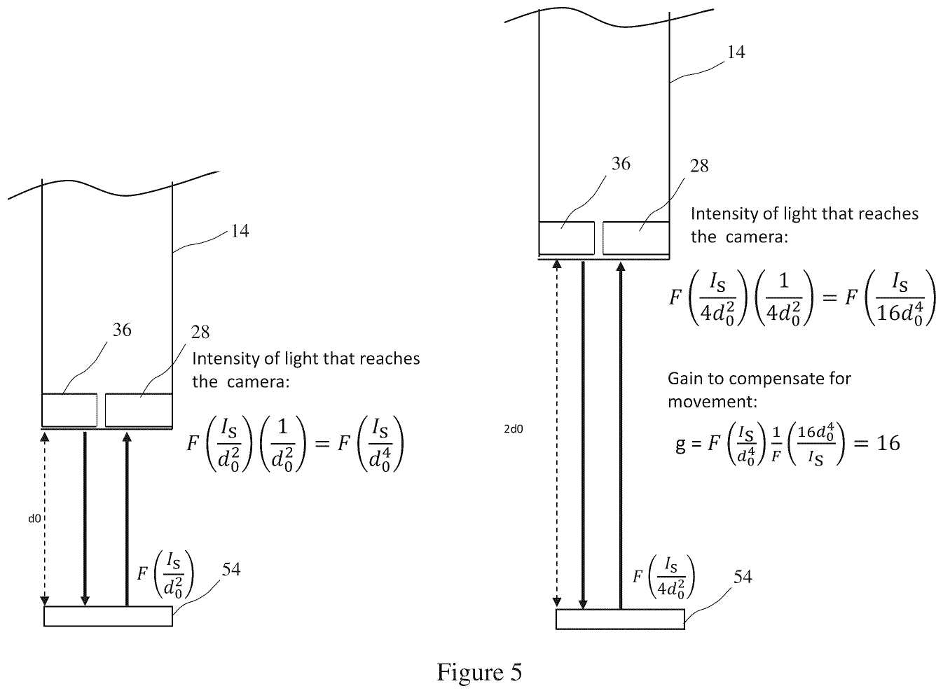

With reference to FIGS. 4 and 5, an endoscopic camera system 10 utilizing a fixed emission reference will now be described. As shown in FIG. 4, the camera 12 may be positioned to visualize an area of interest 50. Within the area of interest 50 may be positioned a fixed emission reference 54. The fixed emission reference 54 may be for example a material painted or impregnated with a fluorescent dye. The fixed emission reference 54 may be selected based on a dye to be used in surgery. The fixed emission reference 54 may be single use or may be sterilizable.

The fixed emission reference 54 may be placed in the area of interest 50 using, for example, an endoscopic forceps, or other grasping device. The fixed emission reference 54 may be placed in the area of interest 50 in a dissolving suture or a dissolving swab. The fixed emission reference 54 may be fixed in the area of interest 50 such as by, for example, a pin or a needle. The fixed emission reference 54 may lay on top of tissue in the area of interest. The fixed emission reference 54 may be an attachment to or mounted in a surgical implement that is positioned in the area of interest. Multiple fixed emission references may be used.

Once the camera 12 and the fixed reference 54 are positioned in the area of interest 50, the camera 12 and the light source 36 may be held fixed and the location of the fixed emission reference within the captured images may be determined. The signal level of the image may be measured at the determined fixed emission reference 54 location. The measured fixed emission reference 54 signal level can change with tissue motion, camera motion, and camera exposure control state, and illumination changes. These four factors may contribute to system exposure changes.

System exposure may be defined as a light intensity impinging upon the image sensor 28 multiplied by a time of sensor integration and multiplied by an exposure control gain. If illumination changes during endoscopy are relatively limited, a change in measured fixed emission reference 54 signal level may provide enough information to compensate the measured fluorescence signal. If the fixed emission reference 54 signal level is L₀ when the camera is not moving, the fixed emission reference is not moving, and the fixed emission reference is at a fixed distance from the camera. This signal level can be determined before fluorescence signals from the area of interest are measured.

After a baseline fixed emission reference 54 signal level is measured, the fluorescence signal measurement may begin. The fixed emission reference 54 signal level at time t during the endoscopy is Lₜ. The ratio

$${g}_{L} = \frac{{L}_{0}}{{L}_{t}}$$

may be a gain that compensates for system exposure changes due to camera motion, tissue motion, and exposure control state changes when multiplied by the measured fluorescence signal.

FIG. 5 illustrates a change in gain when the camera is moved from a first position d₀ to a second position 2d₀ twice as far from the area of interest 50 and the fixed emission reference 54. With the use of a fixed emission reference 54, when the camera 12 is twice as far away, the distance from the excitation light source 36 to both the fixed emission reference 54 and any fluorescence imaging agents used in surgery doubles, which reduces the emitted fluorescence from both the fixed emission reference and the fluorescent imaging agents used in surgery, in addition to a reduction in detected emitted fluorescence as a result of increased distance.

Equations for calculating the relevant gains for non-visible light exposure when the camera is moved from a first position d₀ to a second position 2d₀ twice as far from the area of interest and the fixed emission reference 54 will now be described.

At d₀ the light emitted from the fixed emission reference 54 may be represented by the equation:

$$F ( \frac{{I}_{s}}{{d}_{0}^{2}} )$$

Where F is a fluorescence factor of the fixed emission reference 54 and Iₛ is a brightness of excitation light from the excitation light source 36.

The intensity of light that reaches the camera may be represented by the equation:

$$F ( \frac{{I}_{s}}{{d}_{0}^{2}} ) ( \frac{1}{{d}_{0}^{2}} ) = F ( \frac{{I}_{s}}{{d}_{0}^{4}} )$$

At 2d₀ the intensity of light emitted from the fixed emission reference 54 may be represented by the equation:

$$F ( \frac{{I}_{s}}{4 {d}_{0}^{2}} )$$

At 2d₀ the intensity of light emitted from the fixed emission reference 54 that reaches the camera may be:

$$F ( \frac{{I}_{s}}{4 {d}_{0}^{2}} ) ( \frac{1}{4 {d}_{0}^{2}} ) = F ( \frac{{I}_{s}}{16 {d}_{0}^{4}} )$$

Accordingly, the gain to compensate for this change may be:

$$g = F ( \frac{{I}_{s}}{{d}_{0}^{4}} ) \frac{1}{F} ( \frac{16 {d}_{0}^{4}}{{I}_{s}} ) = 16$$

In additional implementations, in order to account for movement of the camera and a patient, the camera controller 18 may link changes in detected visible light to changes in detected fluorescence. In this implementation, changes in a visible auto-exposure (VIS-AE) index may be directly linked to a non-visible light exposure index (NVL-E). The camera controller may compensate for the scope movement by modifying the NVL-E based on changes in the VIS-AE.

If the intensity changes due to scope distance from the region of interest are approximately the same for visible light as for the fluorescence to be detected, then changes to the visible light exposure index can be used to directly modify the non-visible light exposure index. However, if the intensity changes due to scope distance from the region of interest are different for visible light than for the fluorescence to be detected, then a correction factor can be included to account for changes in intensity.

In an additional implementation, the camera system 10 may modify a non-visible light exposure index based on a combination of changes in a visible light auto-exposure index and a near infra-red (NIR) auto-exposure system. In this implementation, NIR exposure control may be assumed to be independent from visible light exposure control. A fluorescence measurement method, such as for fluorescence emitted from ICG, may start by monitoring a baseline signal for a time interval Δt_{b} and the measured average sensor exposures are e_{b}^{vis} and e_{b}^{nir}. Sensor exposure can be defined as a time of sensor integration multiplied by an exposure control gain.

After a fluorescence imaging agent, such as ICG, is introduced, fluorescence levels may be measured. Motion compensation may comprise one or more of the following steps: a) multiplying the measured fluorescence signal level by the ratio

$${g}_{e}^{nir} = \frac{{e}_{b}^{nir}}{{e}_{t}^{nir}} ,$$

where eₜ^{nir} is the NIR sensor exposure at t, the time of fluorescence measurement, and b) dividing the result by the ratio

$${g}_{e}^{vis} = \frac{{e}_{b}^{vis}}{{e}_{t}^{vis}} .$$

The first step may remove exposure gains corresponding to exposure changes due to scope motion, tissue motion, and fluorescence intensity changes. The second step may return the exposure gains due to scope motion and tissue motion so the resulting compensated signal corresponds to fluorescence intensity changes.

This following table illustrates some examples of a camera system that modifies a non-visible light exposure index based on a combination of changes in the visible auto-exposure index and a near infra-red (NIR) auto-exposure system according to an implementation:

| | Real | | | | | Measured |
|---|---|---|---|---|---|---|
| Example | ICG | Exposure | ge | Exposure | ge | ICG |
| # | intensity | VIS | VIS | NIR | NIR | intensity |
| 1 | 1x | 1x | 1 | 1x | 1 | 1 |
| 2 | 2x | 1x | 1 | 0.5x | 2 | 2 |
| 3 | 2x | 2x | 0.5 | 1x | 1 | 2 |
| 4 | 0.5x | 1x | 1 | 2x | 0.5 | 0.5 |
| 5 | 0.5x | 2x | 0.5 | 4x | 0.25 | 0.5 |

Example 1 illustrates a situation where there is no fluorescence and no motion. The actual fluorescence intensity remains constant (1×). The visible sensor exposure is constant (1×) and the NIR sensor exposure is also constant. Therefore,

$$\frac{{g}_{e}^{nir}}{{g}_{e}^{vis}} = \frac{1}{1} = 1 ,$$

which means the fluorescence measurements remains constant.

Example 2 illustrates a situation where the fluorescence intensity doubles and there is no motion. Since the fluorescence signal doubles, the NIR sensor exposure is halved (which keeps the displayed image brightness constant—a goal of exposure control). Therefore,

$$\frac{{g}_{e}^{nir}}{{g}_{e}^{vis}} = \frac{2}{1} = 2.$$

Example 3 illustrates a situation where the fluorescence signal doubles and the image sensor is moved further away from the area of interest such that the visible and NIR signals fall to half their initial values. In this situation the visible sensor exposure doubles (again to keep the displayed image brightness constant). The increase in fluorescence intensity is counteracted by moving the scope further, so the NIR sensor exposure remains constant. The result is

$$\frac{{g}_{e}^{nir}}{{g}_{e}^{vis}} = \frac{1}{0.5} = 2.$$

Examples 4 illustrates a situation where the fluorescence intensity is halved and there is no motion. The visible sensor exposure stays constant, the NIR sensor exposure is doubled, and the result is

$$\frac{{g}_{e}^{nir}}{{g}_{e}^{vis}} = \frac{0.5}{1} = 0.5$$

Example 5 illustrates a situation where the fluorescence intensity is halved and the image sensor is moved further away from the area of interest such that the signal falls to half its value. The visible sensor exposure doubles, the NIR sensor exposure halves due to the halving of the ICG signal and halves again due to the scope motion—so the resulting NIR sensor exposure is one quarter. The result is

$$\frac{{g}_{e}^{nir}}{{g}_{e}^{vis}} = \frac{0.25}{0.5} = 0.5 .$$

In additional implementations, the camera system may use a visible light exposure index to modify recorded pixel values instead of modifying a non-visible light exposure index. In this implementation an absolute irradiance for each pixel may be estimated by normalizing the recorded pixel value using the visible light exposure index.

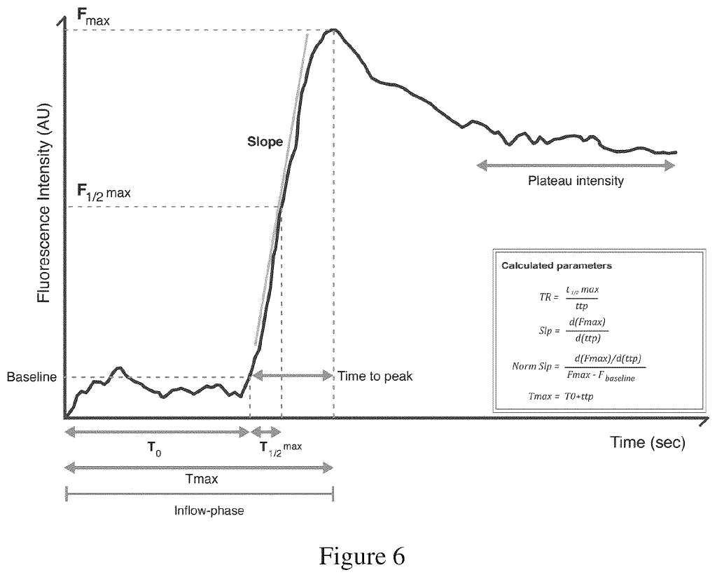

With reference to FIG. 6, administration of a fluorescent dye may be used to assess perfusion in a gastrointestinal tract. In connection with bowel surgery where bowel resection and anastomosis has been performed, fluorescent dye may be administered and a region of interest in the gastrointestinal tract imaged to detect perfusion of the fluorescent dye. Changes in the detected perfusion of the fluorescent dye may be used to identify proper communication between different portions of the gastrointestinal tract and to detect leakage.

In particular, certain parameters can be determined based on image analysis of fluorescent intensity values. For example, a perfusion slope may indicate blood flow in the region of interest. The perfusion slope may be determined from detected fluorescent intensity values within the region of interest over time.

Prior to administration of a fluorescent imaging agent, the detected fluorescence intensity within the region of interest may be an approximately flat line. This may be referred to as the background fluorescence. After administration of the fluorescence imaging agent, the detected fluorescence intensity within the region of interest may rise, because the fluorescence imaging agent may reach the region of interest and begin to fluoresce. This rise may be referred to as the perfusion slope. Typically, the amount of detected fluorescence in the region of interest may rise in a substantially linear manner until a plateau is reached where the amount of detected fluorescence in the region of interest remains relatively stable. At some point the fluorescence imaging agent may start to pass out of the region of interest and the amount of detected fluorescence intensity declines in a washout slope. Various detected parameters may be relevant. For example, the time it takes for the fluorescence imaging agent to flow through may indicate a blood flow speed and may washout slope may add information about organ function.

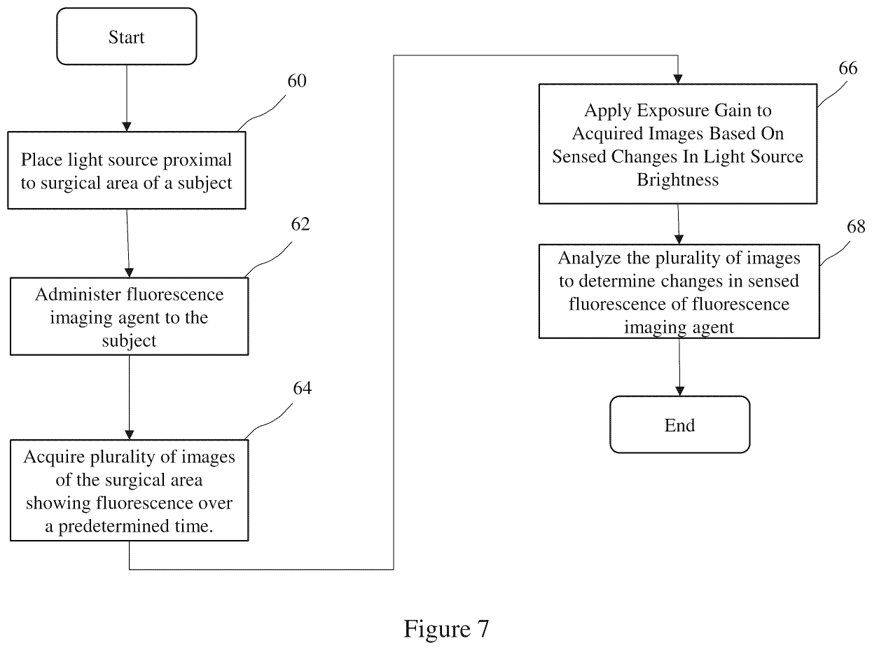

With reference to FIG. 7, a method for fluorescent imaging using an illuminator in a surgical area of a subject will now be described. An illuminator 52 may be placed in a surgical area of a subject, step 60. The illuminator 52 may have an emission band substantially similar to a fluorescence imaging agent to be used. A fluorescence imaging agent may be administered to a patient, step 62. The fluorescence imaging agent may be injected into the patient intravenously.

A plurality of images of the surgical area may be acquired showing fluorescence over a predetermined time, step 64. Exposure gain may be applied to acquired images based on sensed changes in illuminator brightness, step 66. The plurality of images may then be analyzed to determine changes in sensed fluorescence from the fluorescence imaging agent, step 68. In an implementation, the plurality of images may be analyzed to determine a perfusion slope of the fluorescence imaging agent.

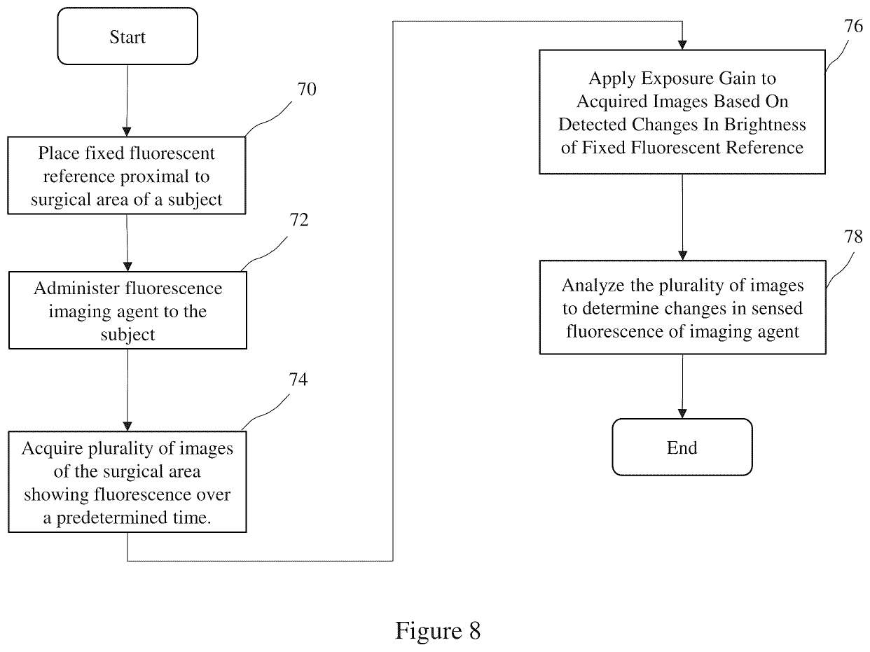

With reference to FIG. 8, a method for fluorescent imaging using a fluorescence reference in a surgical area of a subject will now be described. A fluorescence reference 54 may be placed in a surgical area of a subject, step 70. The fluorescence reference 54 may be selected for having an emission band substantially similar to a fluorescence imaging agent to be used. A fluorescence imaging agent may be administered to a patient, step 72. A plurality of images of the surgical area may be acquired showing fluorescence over a predetermined time, step 74. Exposure gain may be applied to acquired images based on sensed changes in brightness of the fluorescence reference, step 76. The plurality of images may be analyzed to determine changes in sensed fluorescence from the fluorescence imaging agent, step 78. In an implementation, the plurality of images may be analyzed to determine a perfusion slope of the fluorescence imaging agent.

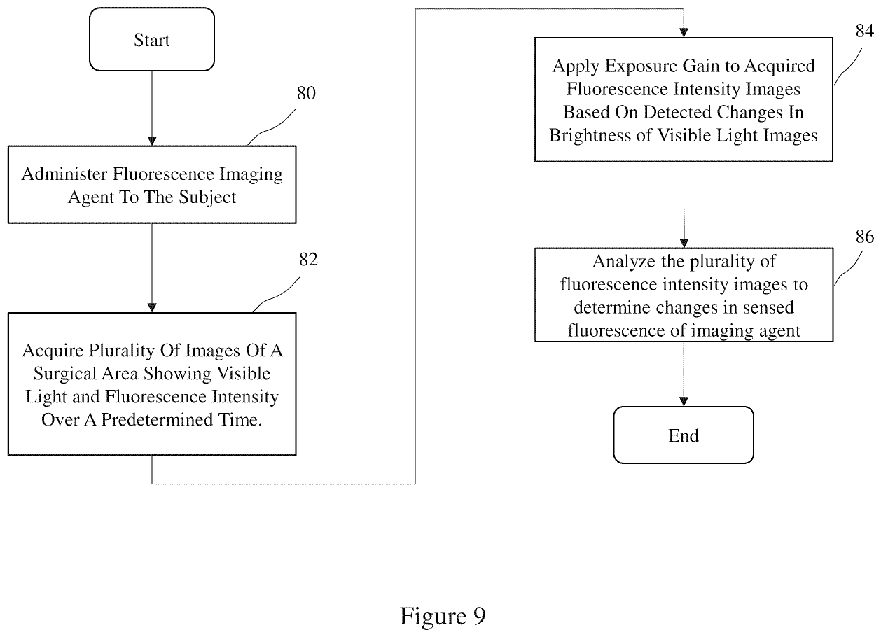

With reference to FIG. 9, a method for fluorescent imaging using detected changes in visible light in a surgical area of a subject will now be described. A fluorescence imaging agent may be administered to a patient, step 80. A plurality of images of the surgical area may be acquired showing visible light and fluorescence intensity over a predetermined time, step 82. Exposure gain may be applied to acquired fluorescence intensity images based on sensed changes in brightness of the visible light images, step 84. The plurality of fluorescence intensity images may be analyzed to determine changes in sensed fluorescence from the fluorescence imaging agent, step 86. In an implementation, the plurality of images may be analyzed to determine a perfusion slope of the fluorescence imaging agent.

Although the systems and methods in this disclosure have illustrated the use of an illuminator 52 and a fixed emission reference 54 for use fluorescence imaging agents, the illuminator 52 and the emission reference 54 may be used with many different wavelengths of imaging agents used in surgery. For example, the illuminator 52 and the fixed emission reference 54 may be, for example, for ultraviolet imaging agents, visible light imaging agents, far infrared imaging agents, and near infrared imaging agents. Additionally, exposure gains may be modified based on specific image sensors.

There is disclosed in the above description and the drawings, a system and method for fluorescence image of a subject that fully and effectively overcomes the disadvantages associated with the prior art. However, it will be apparent that variations and modifications of the disclosed implementations may be made without departing from the principles described herein. The presentation of the implementations herein is offered by way of example only and not limitation.

Any element in a claim that does not explicitly state “means” for performing a specified function or “step” for performing a specified function, should not be interpreted as a “means” or “step” clause as specified in 35 U.S.C. § 112.

What is claimed is:

1. A method for fluorescent imaging of a subject, the method comprising the steps of:
placing an illuminator in a surgical area of a subject, the illuminator having an emission band substantially similar to a fluorescence imaging agent;
administering the fluorescence imaging agent to the subject;
acquiring a plurality of images of the surgical area of the subject showing fluorescence intensity over a predetermined time period; and
analyzing the plurality of images to determine changes in sensed fluorescence from the fluorescence imaging agent;
wherein an exposure gain is applied to the images based on changes in detected intensity of the fluorescence from the illuminator.

2. The method of claim 1 wherein analyzing the plurality of images further comprises determining a perfusion slope of the fluorescence imaging agent.

3. The method of claim 2 wherein the perfusion slope is a direct indication of blood flow in the surgical area of the subject.

4. The method of claim 2 wherein the surgical area is a portion of the gastrointestinal tract of the subject and the perfusion slope is an indication of leakage in the surgical area.

5. The method of claim 1 wherein the fluorescence imaging agent comprises indocyanine green.

6. A method for fluorescent imaging of a subject, the method comprising the steps of:
placing a fluorescence reference in a surgical area of a subject, the fluorescence reference having an emission band substantially similar to a fluorescence imaging agent;
administering the fluorescence imaging agent to the subject;
acquiring a plurality of images of the surgical area of the subject showing fluorescence intensity over a predetermined time period; and
analyzing the plurality of images to determine changes in sensed fluorescence from the fluorescence imaging agent;
wherein an exposure gain is applied to the images based on changes in fluorescence detected from the fluorescence reference.

7. The method of claim 6 wherein analyzing the plurality of images further comprises determining a perfusion slope of the fluorescence imaging agent.

8. The method of claim 7 wherein the perfusion slope is a direct indication of blood flow in the surgical area of the subject.

9. The method of claim 7 wherein the surgical area is a portion of the gastrointestinal tract of the subject and the perfusion slope is an indication of leakage in the surgical area.

10. The method of claim 6 wherein the fluorescence imaging agent comprises indocyanine green.

11. A method for fluorescent imaging of a subject, the method comprising the steps of:
administering a fluorescence imaging agent to the subject;
acquiring a plurality of images of the surgical area of the subject showing visible light and fluorescence intensity over a predetermined time period; and
analyzing the plurality of images to determine changes in sensed fluorescence from the fluorescence imaging agent;
wherein an exposure gain of the fluorescence intensity images is adjusted based on changes detected in the visible light images.

12. The method of claim 11 wherein an autoexposure system provides substantially the same exposure gain adjustment for both the visible light images the fluorescence intensity images.

13. The method of claim 11 wherein analyzing the plurality of images further comprises determining a perfusion slope of the fluorescence imaging agent.

14. The method of claim 13 wherein the perfusion slope is a direct indication of blood flow in the surgical area of the subject.

15. The method of claim 13 wherein the surgical area is a portion of the gastrointestinal tract of the subject and the perfusion slope is an indication of leakage in the surgical area.

16. The method of claim 11 wherein the fluorescence imaging agent comprises indocyanine green.

17. An endoscopic camera system for fluorescent imaging of a subject, the system comprising:
a camera further comprising an image sensor;
a camera controller in communication with the camera, the camera controller further comprising a processor;
a light source coupled to the camera for illuminating a region of interest; and
at least one of the group consisting of: a fluorescence illuminator and a fluorescence reference configured for placement in the region of interest;
wherein the processor is configured to acquire a plurality of images of the region of interest of the subject showing fluorescence intensity over a predetermined time period; analyze the plurality of images to determine changes in sensed fluorescence; and apply an exposure gain to the images based on changes in fluorescence detected from the fluorescence illuminator or the fluorescence reference.

18. The endoscopic camera system of claim 17 further comprising a fluorescence imaging agent; and wherein the processor is further configured to determine a perfusion slope of the fluorescence imaging agent.

19. The endoscopic camera system of claim 18 wherein the fluorescence imaging agent and the fluorescence reference are indocyanine green.

20. The endoscopic camera system of claim 18 wherein the fluorescence imaging agent is indocyanine green and the fluorescence illuminator is configured to emit light in substantially the same wavelength band as indocyanine green.
